(19) United States
(12) Patent Application Publication
(10) Pub. No.: US 2012/0015331 A1
(43) Pub. Date: Jan. 19, 2012

(54) SCAFFOLD

(76) Inventors: Simon Wood, Leeds (GB); Xuebin Yang, Leeds (GB); Marcel de Matas, Bradford (GB); Peter Iddon, Bradford (GB); Michael Raxworthy, York (GB)

(21) Appl. No.: 13/259,084

(22) PCT Filed: Mar. 23, 2010
(86) PCT No.: PCT/GB2010/050485

§ 371 Date: Sep. 22, 2011

(30) Foreign Application Priority Data
Mar. 23, 2009 (GB) 0904907.3

(51) Int. Cl. A61C 19/06; A61K 31/352; A61K 31/5415; A61K 31/409; B29C 35/08; A61K 9/70; A61P 31/04; A61P 31/00; A61P 17/02; A61M 37/00; A61K 9/00; A61K 35/12

(52) U.S. Cl. 433/215; 424/400; 514/454; 514/224.8; 514/410; 424/93.7; 424/402; 424/443; 424/423; 604/20; 264/425

(57) ABSTRACT

The present invention provides a polymeric scaffold containing an antibacterial photoactive drug and optionally comprising seeded cells such as stem cells. The invention also includes methods of using the scaffold for tissue regeneration, prevention or reduction of infection whilst tissue regeneration occurs, methods for improving graft or implant survival, promoting scaffold integration and tissue repair and wound healing.

The present invention relates to a polymeric scaffold support product containing an antibacterial photoactive drug and also to the scaffold product seeded with cells, especially stem cells, methods of using the scaffolds for tissue regeneration and also for the prevention or reduction of infection whilst tissue regeneration occurs. The invention includes inter alia products and methods for improving graft or implant survival, promoting scaffold integration and tissue repair and wound healing. The products and methods of the invention are of particular, but not exclusive, use in regenerative medicine, restorative dentistry, wound management, bone grafting and cosmetic surgery.

### BACKGROUND

Regenerative medicine involves the restoration of diseased, excised and damaged tissue to normal structure and function. It is used in the treatment of a variety of diverse conditions such as wound healing, cancer, infectious diseases, correction of birth defects, musculoskeletal injury and restorative dentistry. Every year, millions of restorative surgical procedures are performed that require donor or replacement tissues to repair damaged or diseased organs and tissues. For example, it is estimated that over 40% of the Western population is lacking one or more teeth and that 5 billion people worldwide (World Health Organization) are affected by dental caries. Advanced bone grafting and regeneration techniques have expanded the possibilities of implant-based restorative dentistry and it is estimated that the use of bone grafts will more than double by 2012. However, a problem associated with all restorative medical procedures is donor material shortages, in order to overcome the lack of availability of natural or native allograft or xenograft tissue for implantation, synthetic non-natural scaffolds have been developed for use in grafting and implantation. Despite the use of such artificial material, even if they are sterilized prior to use/implantation, the problem remains that following any surgical procedure there is a high risk of failure due to infection of the implant itself or the surrounding area, especially the areas directly/indirectly in contact with the environment outside the body. Although systemic or topical antibiotics can be used to combat infection at the site of implantation they may not always be effective, for example the incidence of resistance of bacteria such as MRSA is pandemic and antibiotic therapy is of limited use. In addition it is desirous to avoid overuse of antibiotics to avoid resistance to other strains of bacterial infection.

There is therefore a need for improved scaffolds for use in regenerative medicine that are sterile and that would minimise the likelihood of infection and rejection of the replacement or grafted tissue and that would also have the ability to reduce morbidity to secondary sites.

Photodynamic Therapy (PDT), also known as antibacterial PDT and photodynamic disinfection (PDD), relies upon the principle that a photoactive drug (photosensitiser) is taken up by cells and that when irradiated with light of the appropriate wavelength, the photosensitiser becomes activated and causes cell death/damage by the production of reactive oxygen species (ROS). Neither the light nor the photosensitiser alone is toxic, only when the PDT agent is exposed to light does it become activated and cytotoxic. PDT has been shown to be effective against bacterial, viral and fungal infections.

Erythrosine or erythrosine B is a synthetic xanthene colour additive permitted for use in foods and drugs. It is mainly marketed as the disodium salt of 2′,4,′5,′7′-tetraiodofluorescein. It is a red dye that has an absorption maximum of 530-540 nm in the blue-green range and it is a known photosensitiser/photoactive agent. It has been utilized in both medical and non-medical treatments. Non-medical treatments include insecticidal treatments and industrial surface treatments, and medical treatments include its use as a dye in conjunction with dental treatments so as to visually indicate the presence and location of plaque on teeth, it has also been used in PDT of cancerous and other diseased tissue.

The present invention provides a scaffold encapsulating at least one antimicrobial photoactive agent that can be used in tissue regeneration. It has been found, surprisingly that the scaffold appears to enhance the bactericidal activity of the photoactive agent and that scaffold not only retains that photoactive agent but is also capable of sustained release of said agent acting as a reservoir for prolonged release of the agent. The enhanced bactericidal activity of the scaffold-derived photoactive agent as compared to non-scaffold derived photoactive agent is a completely unexpected finding and may be due to a synergistic effect between the scaffold and photoactive agent.

The present invention uses a novel approach to provide controlled and prolonged localised concentrations of antibacterial therapy at the site of tissue regeneration to prevent infection, prolong scaffold and graft or implant survival and promote successful scaffold integration and tissue repair. It is envisaged that the products and methods of the present invention will maximise the effectiveness of tissue regeneration, minimise infection and improve cosmetic outcomes.

### BRIEF SUMMARY OF THE DISCLOSURE

The present invention provides a scaffold for the co-delivery of an alpha-hydroxy acid and at least one photoactive agent.

According to a first aspect of the invention there is provided a scaffold comprising fibres that provide a source of at least one alpha hydroxy acid and which encapsulate at least one antimicrobial photoactive agent.

Alpha hydroxy acids include, for example and without limitation, glycolic acid, lactic acid, citric acid, mandelic acid, tartaric acid, malic acid, and galacturonic acid. Preferably, the alpha hydroxy acid is glycolic acid.

Reference herein to “fibres that provide a source” of an alpha hydroxy acid include fibres that can generate an alpha hydroxy acid by degradation of the fibre by for example hydrolysis or fibres that are composed of an alpha hydroxy acid or fibres that are coated with or covered in an alpha hydroxy acid.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Reference herein to a “scaffold” is intended to include a porous, three dimensional network or mesh of randomly orientated or aligned fibres which is capable of supporting or encapsulating an agent or cells therein and/or thereon.

Reference herein to the scaffold “containing” a photoactive agent includes encapsulating or trapping or loading or retaining or coating or absorbing or adsorbing the photoactive agent within and/or on the surface of its fibrous mesh/network. The preferred embodiment is where the photoactive agent is encapsulated within the fibrous network of the scaffold thereby controlling the release of the photoactive agent from the scaffold.

Reference herein to cells being “seeded therein or thereon” is intended to include cells encapsulated or entrapped or loaded or retained or adhered or captured or anchored to the scaffold surface or within its fibrous mesh/network. The cells being derived from a selected cell source material.

In preferred embodiments of the invention the scaffold releases the alpha hydroxy acid for example glycolic acid from the fibres as a product of hydrolysis and so preferably, the fibres are composed of a biocompatible polymer such as poly(glycolic acid) (PGA) or a copolymer thereof. Other suitable polymers that release alpha-hydroxy acid as a product of hydrolysis are also included in the scope of the invention and include, for example and without limitation, α-hydroxy acid-based polymers such as poly(lactic-co-glycolic acid) (PLGA) or a copolymer thereof or poly(lactic acid) (PLA) or a copolymer thereof, blends and mixtures with each other or with PGA. In other embodiments of the invention the fibres may comprise any alternative synthetic or natural polymer. The term “synthetic polymer” means any polymers that are not found in nature, even if the polymers are made from naturally occurring biomaterials. Examples include, but are not limited to, aliphatic polyesters, poly(amino acids), polyetheresters, polyalkenes, polyurethanes, polyamides, tyrosine-derived polycarbonates, poly(imino carbonates), polyorthoesters, polyoxaesters, polyoxaesters containing amino groups, polyamidoesters, polyanhydrides, polyphosphazenes and combinations thereof. The term “natural polymer” refers to any polymers that are naturally occurring, for example, silk, collagen-based materials, fibrin, chitosan, hyaluronic acid and alginate and combinations thereof. Preferably, the fibres are composed of bioresorbable or bioabsorbable material.

Examples include, but are not limited to, collagen, fibrin, hyaluronic acid, poly(lactic acid), poly(glycolic acid), polycaprolactone, polydioxanone, poly(trimethylene carbonate), poly(ethylene glycol), alginate, chitosan or mixtures thereof. Accordingly and advantageously, the bioresorbable or bioabsorbable nature of the scaffold material means that it does not require subsequent removal following implantation, although in some embodiments of the invention it may be desired to use non-biodegradable fibres to provide a more permanent support for the seeded cells to grow upon. For example, where the scaffold material is used for abdominal hernia repair, the long-term retention of tensile strength afforded by non-biodegradable fibres or filaments may help prevent reoccurrence of the hernia.

Reference herein to “implantation” is intended to include placement either within the body or on a surface of the body for example skin or buccal cavity oral mucosa.

In one specific embodiment of the invention the fibres of the scaffold comprise electrospun PGA, so that the scaffold may eventually be broken down in situ by hydrolysis, the resulting by-products being metabolised by normal biochemical pathways and ultimately lost during respiration as carbon dioxide and water. As mentioned hereinbefore other polymers that release glycolic acid as a product of hydrolysis are also suitable to be used in the present invention.

Preferably, the fibres of the scaffold may comprise a mixture of polymers or copolymers that release glycolic acid as a product of hydrolysis so that the fibres may comprise PGA and/or PGLA. The fibres may also comprise a mixture of polymers or copolymers that release glycolic acid as a product of hydrolysis optionally in addition to including natural fibres. The scaffold may therefore comprise a mixture of fibres composed of different polymers or copolymers of different molecular weight and/or of different diameters. Such embodiments allow for different rates of release of the photoactive agent, for example the scaffold may be constructed to provide an initial burst or high rate of release of the photoactive agent followed by a second slower sustained release over a protracted period. Alternatively, the scaffold may be constructed to provide pulsed or alternative releases of the photoactive agent such as high, low, high, low release rates. In one embodiment of the invention, monomeric glycolic acid is co-administered from the scaffold.

Preferably, the scaffold contains at least one photoactive agent or it may comprise a mixture of different photoactive agents. The photoactive agent(s) to be incorporated into the scaffold should ideally be a pure, well-defined compound with spectral characteristics which would allow excitation with light (either laser or non-coherent white light) in the visible region of the electromagnetic spectrum preferably, but not necessarily, at longer wavelengths which would increase tissue penetration of the light. It should be released from the scaffold in a well-defined manner over a time period which is relevant to infection times in vivo (3-14 days). The photoactive agent should be non-toxic to cells in the absence of light, and should specifically kill bacteria upon irradiation by the appropriate use of drug dosimetry. Examples of suitable photosensitisers that may be used with the scaffolds and methods of the present invention include, but are not limited to: xanthenes (eg. erythrosine), porphyrins (eg. haematoporphyrin), phthalocyanines (eg. sulfonated aluminium phthalocyanine), chlorins (eg. tin IV chlorin e₆) and thiazines (eg. methylene blue, toluidine blue).

Accordingly, a particularly preferred photoactive agent of the present invention is selected from the group comprising erythrosine B, methylene blue, polychrome methylene blue, haematoporphyrin IX and chlorine e₆.

Preferably, the scaffold comprises between 0.1 to 20.0% w/w of the photoactive agent and more preferably between 1.0 to 10% w/w. The degree of loading is likely to be commensurate with the specific application. It will also be appreciated that the scaffold may be loaded with different concentrations of a photoactive agent in different areas of the scaffold or indeed the scaffold may be loaded with different concentrations of different agents in different areas of the scaffold.

Preferably, the scaffold of the present invention provides a means by which a combination of a photoactive agent and glycolic acid can be delivered to a desired site. Results have shown that there is a synergistic effect between the photoactive agent and glycolic acid. It is also reasonable to expect that such synergy would occur with other alpha-hydroxy acids such as lactic acid. Accordingly, the scaffold of the present invention allows for delivery of for example an alpha hydroxy acid such as a glycolic acid monomer (which may be derived as a product of hydrolysis from the polymeric fibres or may be directly co-administered or maybe directly derivable from the scaffold as the monomer) plus a photoactive agent that is released from the scaffold, to a desired site.

Preferably, the scaffold is seeded with a population of cells, preferably stem cells, progenitor cells or stem cell containing population and more preferably it is seeded with human dental pulp stem cells (HDPSCs). It will be appreciated that the scaffold of the present invention may also be seeded with differentiated cells or a mixture of differentiated and undifferentiated cells and that the cells can be derived from any cell or tissue source. For example and without limitation, cell types that may be seeded onto or into the scaffold include: mesenchymal or stromal cells such as fibroblasts, smooth muscle cells, tenocytes, ligament cells, osteoblasts, skeletal or cardiac myocytes, reticulo-endothelial cells and chondrocytes; neuroectodermal cells such as neurons, glial cells or astrocytes, endocrine cells (such as melanocytes, or adrenal, pituitory, or islet cells), blood cells such as platelets, leukocytes and/or their progenitors, the type of cell seeded into or onto the scaffold of the present invention is not intended to limit the scope of the application. Accordingly, once the scaffold is seeded with cells then depending on the type of cells contained within the scaffold the uses of the scaffold may be applicable to many areas of medicine and therapy.

Preferably, the scaffold is a non-woven fabric.

Preferably, the fibres have a mean fibre diameter of between from about 0.01 to 100.00 microns.

It will be appreciated that the fibre diameters are in the range of microns to nanometers. Preferably, the mean fibre diameter is between 0.05 and 50.00 microns or 0.10 and 10 microns. It will be appreciated that the mean fibre diameter is selected according to a user's requirements and the cell type which is to infiltrate into the mesh network and that, in some instances, it is desired to provide larger fibre diameters so that cells can migrate into the scaffold. It may also be desirable to provide a mixture of fibres of different diameters.

The scaffold may preferably be in the form of a sheet or strip or patch or alternatively it may be in a form that is deliverable by aerosol or injection so that it is formed in situ at the site of application. The scaffold product may also be provided in other forms such as and without limitation, rods, blocks, spheres or may be incorporated into bandages and such like.

In further embodiments of the invention the scaffold is manufactured by electrospinning (either solution or melt electrospinning), phase separation, melt-blowing, spinning or self-assembly.

According to a second aspect of the invention there is provided a method of manufacturing a scaffold comprising electrospinning a solution comprising a biocompatible polymer and a photoactive agent onto a target, wherein the electrospun fibres have a mean fibre diameter of between from about 0.01 to 100.00 microns so as to form a scaffold, optionally the method further comprising the step of seeding the scaffold with a population of cells.

According to a third aspect of the invention there is provided a scaffold obtainable by the method of the second aspect of the invention for use in tissue engineering, tissue repair, cosmetic or reconstructive surgery, reconstructive surgery of congenital birth defects, wound healing, wound repair, improving graft or implant survival, promoting scaffold integration, as augmentation material in surgery or for the implantation of cells into a host in need of therapy.

According to a fourth aspect of the invention there is provided a method of delivering a selected population of cells to a tissue comprising implanting the scaffold of the first aspect of the invention when seeded with a selected population of cells at an implantation site.

As mentioned herein before the type of cells that are seeded on to or into the scaffold will dictate the area of medicine or therapy for which the scaffold may be used.

According to a fifth aspect of the invention there is provided a method of reducing or controlling the risk of microbial infection following implantation of a scaffold according to the first aspect of the invention, the method comprising implanting the scaffold of the first aspect of the invention at an appropriate site and exposing it to light so as to activate the photoactive agent.

Reference herein to a “microbial infection” includes bacterial, fungal and viral infections.

As stated hereinbefore, the scaffold may optionally be seeded with a selected population of cells.

According to a sixth aspect of the invention there is provided a method of improving graft or implant survival and/or promoting scaffold integration and/or tissue repair and/or wound healing the method comprising implanting the scaffold of the first aspect of the invention at an appropriate site and exposing it to light so as to activate the photoactive agent.

Preferably, the methods of the invention comprise subjecting the scaffold containing the photoactive agent to a single or periodic burst(s) of light so as to activate the agent. The duration and frequency of light exposure is selected according to a user's requirements. In the instance of using erythrosine B the wavelength of light required for activation would be around 530-540 nm i.e. in the blue-green range of visible light. Accordingly, the methods of the fourth and fifth and sixth aspects of the invention are suitable for implantation at any site on the surface or within the human or animal body that is capable of receiving a light source. For example the implantation site may be selected from the group comprising, skin, buccal/oral cavity, gastro-intestinal tract, upper respiratory tract, pulmonary airways, urino-genital tract, abdomino-pelvic region, auditory passages, joints and in some instances may be subcutaneous for example in and around mammary glands.

It will be appreciated that the present invention provides inter alia a fibrous non-woven scaffold comprising electrospun fibres of a bioresorbable or bioabsorbable polymer and a photoactive agent such as erythrosine B. Once placed in/on a wound or somewhere within the body of a human or animal, the highly porous scaffold will release erythrosine B, which is taken up by microbes such as bacteria. Erythrosine B is then activated by light and selectively causes bacterial death. Light or photosensitiser acting alone are both non-toxic. The effectiveness of the photosensitiser in killing bacteria is surprisingly greater when released from the scaffold than when used alone, without wishing to be bound to scientific theory, it is believed that there is a synergistic effect of photoactive agent and scaffold. Also advantageously, the scaffold can be seeded with cells which are not affected by the presence of the photoactive agent so that they may be allowed to proliferate in situ in or on the body and establish themselves and generate new tissue without risk of infection at the site and rejection of the scaffold.

Advantageously, the scaffold of the invention acts as a temporary reservoir for delivery of erythrosine B or similar products to prevent/treat microbial infections and/or support tissue regeneration. Subsequent PDT provides a means for removing for example bacteria from a wound site, minimising the rate of failure and associated complications.

According to a seventh aspect of the invention there is provided a method of restorative dentistry comprising implanting the scaffold of the first aspect of the invention seeded with human dental pulp stem cells at an appropriate site within the buccal cavity and exposing it to light so as to activate the photoactive agent.

It will be appreciated that the seventh aspect of the invention provides a convenient and improved method of tissue regeneration in restorative dentistry for teeth, bone and any other tissue in the oral cavity and a significant contribution to the art. In the embodiment of the scaffold being bioabsorbable or bioresorbable the scaffold will remain in situ in the mouth releasing the photoactive agent over a period of time, typically at least 5 days, whilst allowing the seeded cells to establish and grow without impedance of a bacterial infection.

Other healthcare benefits of the present invention include: highly efficacious treatment allowing cost and time savings for health systems globally; a convenient solution to infection resistance since bacterial infections are controlled at an early stage; reduction in the need for the use of antibiotics; improved cosmetic outcome/improvement in patient quality of life; prolonged and improved construct survival reducing the need for revision surgery.

The healthcare benefits noted above are likely to have a significant health economic impact. They are likely to lead to reduced numbers of hospital visits and also importantly a reduced chance of infection and hence reduced associated costs.

According to an eighth aspect of the invention there is provided a method of controlled release of a photoactive agent at a specified site in or on a human or animal body comprising implanting the scaffold of the first aspect of the invention, optionally seeded with a selected population of cells at or in said specified site.

According to a ninth aspect of the invention there is provided a scaffold for delivering a glycolic acid monomer and at least one photoactive agent to a desired site, wherein the glycolic acid monomer is derived as a product of hydrolysis from polymeric fibres within said scaffold or is directly co-administered or is directly derivable from scaffold fibres as the monomer.

It will be appreciated that the preferred features ascribed to the first aspect of the invention apply mutatis mutandis to the second, third, fourth, fifth, sixth, seventh, eighth and ninth aspects of the invention and that all preferred features are equally applicable to each and every aspect of the invention.

The invention is further illustrated by the following figures and examples, but is not limited thereby.

### BRIEF DESCRIPTION OF THE DRAWINGS

FIG. 1 shows a scanning electron microscope image of the fibrous PGA scaffold containing erythrosine, the scale bar corresponds to a length of 10 μm.

FIG. 2 shows the cumulative erythrosine release from a scaffold immersed in distilled water, phosphate buffered saline (PBS) or PBS and foetal calf serum (FCS) at either 5% or 10% and incubated at 37° C. over a total of 8 days. Error Bars represent one standard error from the mean (n=5)

FIG. 3 shows erythrosine concentration at 2, 6, 10 and 14 mm from edge of scaffold disk(s) diffused into surrounding agar.

FIG. 4 shows HDPSC grown on scaffold stained with phalloidin Alexaflour 488. FIG. 4A shows the cells 1 day after seeding, FIG. 4B after 4 days, FIG. 4C after 5 days and FIG. 4C after 6 days.

FIG. 5 shows the number of colony forming units of L. Casei formed after 0, 10 or 30 minute irradiation in BHI broth with either no additives, scaffold breakdown product “blank scaffold”, 22 μM non scaffold derived erythrosine or 22 μM scaffold derived erythrosine (n=6).

FIG. 6 shows HDPSC survival over irradiation time following PDT treatment in the presence of scaffold-derived erythrosine (n=6).

FIG. 7 shows HDPSC survival over irradiation time following PDT treatment in presence of non scaffold derived erythrosine (n=6).

FIG. 8 shows a scanning electron microscope image of the fibrous PGA scaffold containing 5% methylene blue, the scale bar corresponds to a length of 100 μm. The measured mean fibre diameter is 1.31 μm.

FIG. 9 shows a scanning electron microscope image of the fibrous PGA scaffold containing 10% methylene blue, the scale bar corresponds to a length of 100 μm. The measured mean fibre diameter is 1.23 μm.

FIG. 10 shows a scanning electron microscope image of the fibrous PGA scaffold containing polychrome methylene blue, the scale bar corresponds to a length of 100 μm. The measured mean fibre diameter is 0.91 μm.

FIG. 11 shows a scanning electron microscope image of the fibrous PGA scaffold containing toluidine blue O, the scale bar corresponds to a length of 100 μm. The measured mean fibre diameter is 0.74 μm.

FIG. 12 shows a scanning electron microscope image of the fibrous PGA scaffold containing haematoporphyrin IX, the scale bar corresponds to a length of 100 μm. The measured mean fibre diameter is 0.83 μm.

FIG. 13 shows a scanning electron microscope image of the fibrous PGA scaffold containing chlorin e₆, the scale bar corresponds to a length of 100 μm. The measured mean fibre diameter is 0.75 μm.

FIG. 14 shows a scanning electron microscope image of the fibrous PLGA 10:90 scaffold containing methylene blue, the scale bar corresponds to a length of 100 μm. The measured mean fibre diameter is 0.77 μm.

FIG. 15 shows a scanning electron microscope image of the fibrous PLLA scaffold containing methylene blue, the scale bar corresponds to a length of 100 μm. The measured mean fibre diameter is 0.74 μm.

FIG. 16 shows a scanning electron microscope image of the fibrous PCL scaffold containing methylene blue, the scale bar corresponds to a length of 100 μm. The measured mean fibre diameter is 0.20 μm.

FIG. 17 shows photographs of the fibrous dye-containing scaffolds, showing a control PGA scaffold with no dye (A); a PGA scaffold containing 5% erythrosine B (B); a PGA scaffold containing 5% methylene blue (C); a PGA scaffold containing 5% polychrome methylene blue (D); a PGA scaffold containing 5% toluidine blue O (E); a PLGA scaffold containing 5% methylene blue (F); a PLLA scaffold containing 5% methylene blue (G); a PCL scaffold containing 5% methylene blue (H); a PGA scaffold containing 10% methylene blue (I); a PGA scaffold containing 5.75% haematoporphyrin IX (J);and a PGA scaffold containing 2.15% chlorin e₆ (K).

FIG. 18 shows the amount of glycolic acid/erythrosine released from a PGA scaffold in PBS at room temperature (FIGS. 18A and 18B) at 37° C. (FIG. 18C and FIG. 18D).

### DETAILED DESCRIPTION

### Scaffold Preparation

Polyglycolic acid (PGA) was melt-extruded at 260-274° C. using a single screw extruder and then immediately quenched in water at 5-10° C. This extruded PGA was then vacuum-dried and stored at −18 ° C. This extruded PGA was then used to prepare 12.0 w/w % solutions of PGA in hexafluoroisopropanol (HFIP) containing 5.0 w/w % erythrosine B (sodium salt) relative to the dry weight of PGA. PGA and erythrosine B (sodium salt) were weighed into a glass vial and left until dissolved. Prior to electrospinning, the solution of PGA and erythrosine B in HFIP was filtered through a 10 μm polypropylene filter into a polypropylene syringe. The resulting clear red solution was then loaded into a syringe pump.

The syringe exit was connected to a HFIP-resistant flexible plastic tube, which then split into two tubes. These tubes connected to two flat-ended 21 gauge steel needles, which were supported in a needle arm which could be made to traverse by means of a motor. The needles were aligned perpendicularly with respect to the rotational axis of the earthed 50 mm diameter, 200 mm long steel mandrel and the needle tip to mandrel separation distance was set to 60 mm. The needles were set to traverse along the entire 200 mm length of the mandrel, at a rate of one traverse every 18.5 seconds (where a traverse is defined as a single movement forward or backward along the length of the traversing distance). The syringe pump was set to dispense polymer solution at 0.06 mLmin-1 (0.03 mLmin-1 per needle). The mandrel was completely covered in a sheet of non-stick release paper (fastened in place using double-sided adhesive tape) and rotated at 50 rpm by means of a motor. A voltage of 15.0 kV was delivered to the needles.

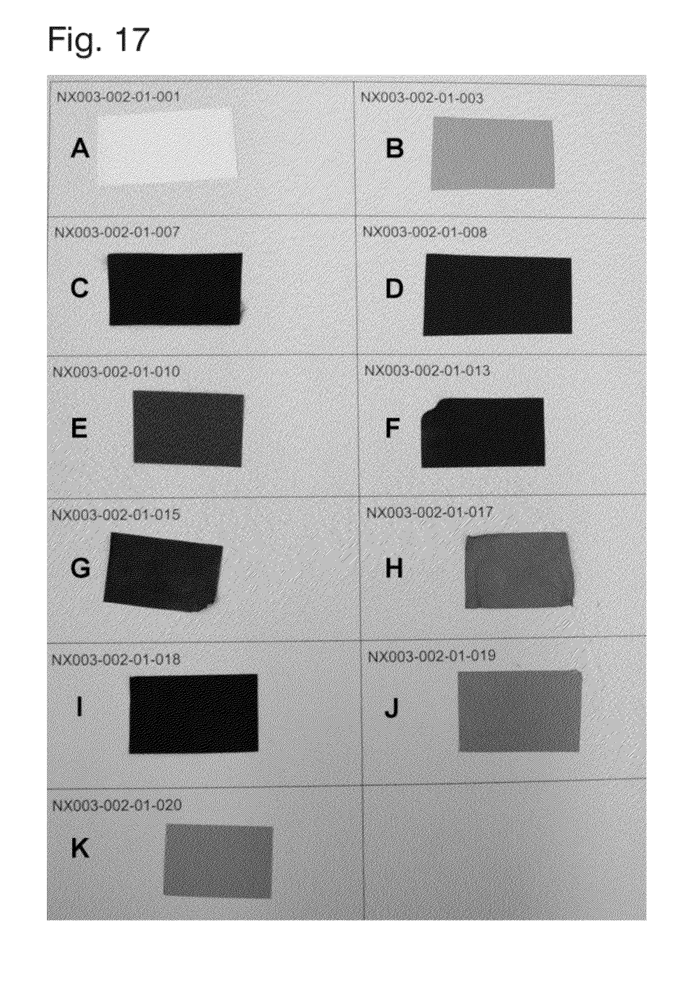

Electrospun fibres were then formed from the solution of PGA and erythrosine B delivered to the needle tips, and collected on the paper-covered mandrel to form a non-woven scaffold material. Electrospinning was carried out at 21±1° C. After a period of 55 minutes, the voltage generator was switched off and the scaffold removed from the mandrel. The scaffold was then dried overnight in a vacuum oven at room temperature, to remove any residual HFIP. The thickness of the single scaffold layer produced was measured at several points along its length (i.e. parallel to the rotational axis of the mandrel) using digital calipers. The thickness of this scaffold was determined to be 100-110 μm along the central portion of the scaffold (75-80%). FIG. 17 shows a photograph of the scaffold obtained (labeled B) compared to a control scaffold not containing any erthyrosine B (labeled A).

### Scanning Electron Microscopy (SEM)

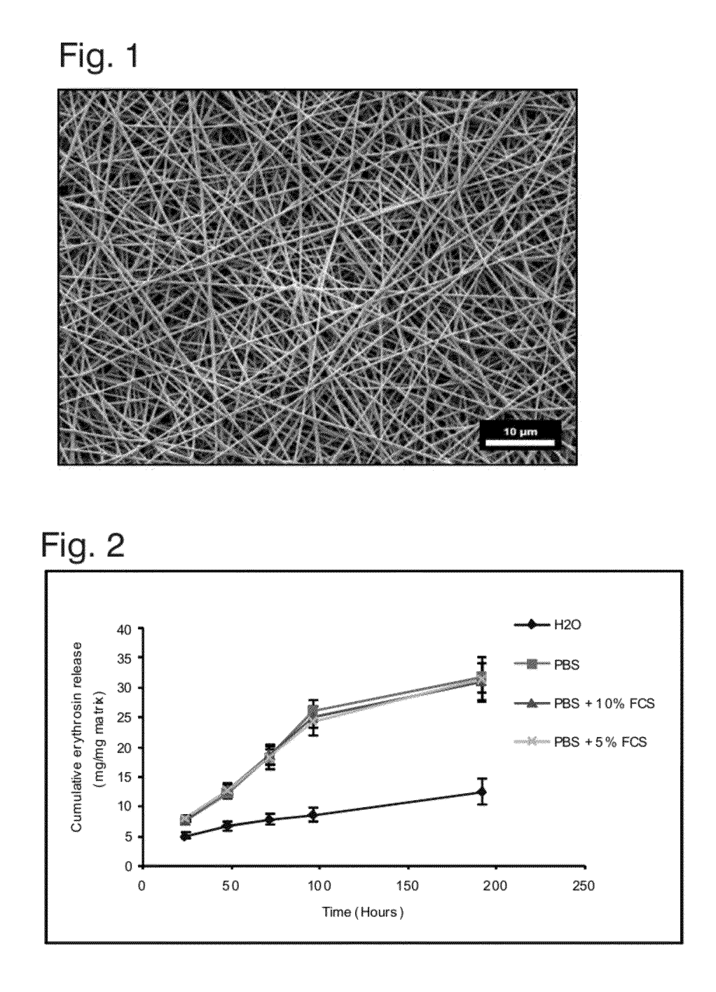

Electrospun scaffolds were dried under vacuum overnight prior to SEM analysis. Samples were attached to 12 mm aluminium SEM stubs using two small pieces of double-sided adhesive to either edge, leaving a central zone without adhesive. The samples were attached so that the upper surface of the scaffold was visible (i.e. the surface deposited towards the end of the experiment). Samples were then sputter coated with gold/palladium alloy to an estimated depth of approximately 30 nm. The coated samples were subsequently imaged by an FEI-Quanta Inspect SEM in the high vacuum mode using a voltage of 5.0 kV and spot diameter of 2.5 nm. A typical SEM image acquired at a magnification of 4,000 is shown in FIG. 1. Three SEM images at a suitable magnification were recorded and printed for one sample of each electrospun fibre scaffold, and these were used to calculate the mean fibre diameter. For each image, the diameters of the first 20 clearly visible fibres along a randomly selected straight line were measured using a ruler. The aggregate 60 measurements from the three images were used to calculate a mean fibre diameter and standard deviation. The mean fibre diameter for this scaffold was determined to be 0.38 μm with a standard deviation of 0.06 μm.

Extraction of Erythrosine B from Scaffolds

Two extraction methods were employed for the removal of erythrosine B from the polymeric scaffolds. The first method used phosphate buffered saline (PBS). A small section of the scaffold was cut and placed in 10 ml of PBS for approx 14 days. After the specified time interval, 1.5 ml of the solution was removed and analysed. For the second method (which enables a more rapid extraction of erythrosine B from the scaffold), a small section of scaffold was cut and placed into the bottom of a 5 ml, glass, flat bottomed vial. 2 ml of a 5% solution of ammonia solution was added. The sample was left for 1 hour, after which a 0.5 ml aliquot was removed from the vial, diluted in 1 ml of methanol and analysed.

Erythrosine Release from Scaffold into Liquid

1 cm² pieces of scaffold were prepared and individually weighed and placed in a well of a 12 well tissue culture plate well containing either distilled water-PBS pH 7.4, PBS plus 5% or 10% foetal calf serum (FCS) and a distilled water control. Samples were incubated at 37° C. for 24 hours following incubation solution was aspirated; stored and fresh solution was added. This was repeated to give a total of four 24 hour periods. After removing the solution on the fourth day fresh solution was added and incubated at 37° C. for a further 96 hours after which the solution was aspirated and stored. The concentration of erythrosine in each solution at each time point was determined by visible light spectrophotometry, this is possible due to the spectroscopic characteristics of erythrosine. In aqueous solution erythrosine absorbs visible light and has an absorption maxima of approximately 540 nm. Measurement was carried out using a Shimadzu UV-2401PC UV-visible light spectrophotometer.

Erythrosine Release from Scaffold into Gelatinous Medium

1 cm² square of scaffold was first embedded into a nutrient agar to confirm erythrosine was able to diffuse through solid agar. 6 mm diameter disks of scaffold were then prepared and an average weight was recorded these were then sterilised by immersion in 70% ethanol followed by drying in aseptic conditions. Brain heart infusion (BHI, Oxoid) agar was prepared in a molten state and poured to half depth into Petri dishes. A number of scaffold disks were laid on top of the agar such that scaffold disks were stacked vertically in the centre of the Petri dish. A second layer of agar was then added to the Petri dish thereby totally covering the scaffold disks. Petri dishes were then incubated at 37° C. for 24 hours. 3 mm diameter cores were extracted from the agar starting from 2 mm outside of the periphery of the scaffold disks at 4 mm intervals, these cores were then added to PBS and heated to re-melt the agar. Once dissolved the concentration of erythrosine was measured using the Shimadzu UV-2401PC UV-visible light spectrophotometer.

Human Dental Pulp Tissue Preparation; Stem/Stromal Cells (HDPSCs) Isolation and in vitro Expansion

Teeth were obtained with patients' informed consent following extraction. Human dental pulp was extracted from sound intact teeth, which had been surgically removed for clinical reasons. Each tooth was washed within a Class II hood and cracked in a bench vice. The dental pulp tissues were harvested and washed with 1×PBS and minced into small pieces (1×2×2 mm3) which were kept in the PBS and ready for use. The HDPSCs were isolated from human dental pulp tissues using organ culture methods or collagenase digestion.

### Growth of Human Dental Pulp Stem Cells (HDPSCs) on Scaffold

HDPSCs were harvested and grown in a monolayer culture to confluency in standard Dulbecco's Modified Eagle Medium (DMEM, Sigma) in supplement of 10% FCS plus penicillin/streptomycin at 37° C., 5% CO₂. Cells were typsinised and re-seeded in at a density of 1×10⁵ cells per scaffold sample. Scaffold was secured by minusheet® clips in a single well of a 24 well tissue culture plate. Cells were incubated and then taken out of incubation after a number of days, fixed with 10% neutral buffered formalin and stained by incubation with phalloidin conjugated to alexaflour 488 (Invitrogen) which binds to cytoskeletal actin. Images were then observed and recorded using Leica TCS SP2 confocal microscope.

Bacterial Killing Assay

L. Casei is a known constituent of gut microflora. A growth curve for L. Casei both in the presence and without erythrosine was prepared using broth turbidity measured by spectrophotometry using a Shimadzu UV-1601 UV-Visible spectrophotometer. L. Casei were grown in Brain Heart Infusion (BHI) liquid broth at 37° C. to stationary phase overnight. A fresh volume of BHI broth containing erythrosine released from the scaffold at a concentration known to be effective for PDT was inoculated using the stationary phase culture and incubated at 37° C. in a shaking incubator. Bacteria were also incubated in BHI with non scaffold derived erythrosine, BHI containing an equal concentration of scaffold breakdown products from a non erythrosine containing scaffold or blank scaffold and BHI alone with no additives. Since bacteria are most sensitive to PDT during log phase this was chosen as the time most appropriate for irradiation therefore irradiation took place 2 hours 30 minutes after inoculation. Irradiation was performed as described by Wood et al., 2006 J Antimicrob Chemother 57 (4): 680-4, by using a 400 W tungsten filament lamp suspended at a distance of 30 cm with a heat dissipating water bath between lamp and broth sample. The output of the lamp was 22.5 mW/cm² in the wavelength range 500-550 nm. Samples were irradiated over a period of 0, 10 or 30 minutes. Following irradiation bacterial viability was assessed by spreading broth samples on Columbia blood agar plates and following 48 hour incubation counting the number of visible colonies. The number of colony forming units (cfu)/ml of broth were then calculated.

### Mammalian Cell Killing Assay

HDPSCs were grown in a monolayer culture to confluency in standard Dulbecco's Modified Eagle Medium (DMEM) plus 10% FCS plus penicillin/streptomycin at 37° C., 5% CO₂. Cells were trypsinised and re-seeded at a density of 1×10⁴ cells/well into a 96 well tissue culture plate. Cells were incubated as before overnight then media was replaced with fresh media plus non scaffold derived erythrosine or media that had been conditioned by overnight incubation at 37° C. in the presence of either scaffold or a control blank scaffold containing no erythrosine and cells were returned to the incubator for 2 hours 30 minutes (this incubation time was used to keep consistency between bacterial and mammalian killing assays). Following incubation cells were irradiated as described previously for 0, 5, 10, 20 and 30 minutes. After washing with PBS to remove residual erythrosine cells were incubated as before with normal DMEM+10% FCS+P/S overnight. Cell survival was assessed using the Cell Titre 96 AQᵤₑₒᵤₛ One Solution Cell Proliferation assay (Fisher) which utilises cellular NADPH or NADH to convert a tetrazolium compound into a Formazan product that can be detected by its absorbance at 490 nm. After incubation of cells with tetrazolium compound diluted in culture medium absorbance was read using a MRX II microplate reader.

### EXAMPLE 1

Experiments were conducted to determine the active agent content of the scaffold and to assess the chemical stability using a reverse phase HPLC method for the determination of erythrosine levels in polymer scaffold samples. FIG. 1 shows a scanning electron microscope image of the fibrous PGA scaffold, the scale bar corresponds to a length of 10 μm.

In order to determine the quantity of erythrosine encapsulated within the manufactured scaffolds, two methods for extracting erythrosine were employed. Using the PBS method hereinbefore described, it is clear that PBS is able to effectively extract erythrosine from the scaffolds giving an average value of active agent content of 5% w/w for each reference sample. However, a minimum of 2 weeks is required for extraction of the entire contents of the scaffold, which can be observed by the colour change of the samples from pink to white. Using the more rapid ammonia extraction method, in which the scaffolds were immersed in a solution of ammonia (1% v/v), the results also showed that the loading of erythrosine in the scaffolds was approximately 5% w/w per scaffold sample. These data demonstrate that both extraction methods can be used to quantify the loading of erythrosine in the scaffold samples and that both methods indicated an approximate erythrosine content of 5% w/w for the reference material. This erythrosine loading equates to the nominal level of erythrosine introduced into the electrospun materials, which demonstrates an encapsulation efficiency of 100%. Negligible losses of erythrosine therefore occur during the electrospinning process.

Regarding stability, portions of the loaded scaffold were placed in glass vials and left on the bench top for 4 weeks to assess the stability of the product at room temperature (20-25° C.). To ascertain the impact of light exposure, selected samples were protected with aluminium foil, whilst others were exposed to natural light. All stability analyses were conducted in duplicate. Reference samples were stored under refrigerated conditions at 5° C. The data (not shown) indicates that the erythrosine content was maintained when samples were stored under refrigerated conditions and when stored at room temperature protected from light for a period of 4 weeks. However, there was notable loss of active agent content, when samples were stored at room temperature and exposed to light.

In conclusion, the data generated demonstrate that the electrospinning process is able to produce scaffolds with 100% encapsulation efficiency, giving an erythrosine loading of approximately 5% w/w. Stability analysis has shown that samples are stable when stored at room temperature for 4 weeks, except for those scaffold samples exposed to light.

### EXAMPLE 2

Experiments were conducted to quantify the amount of erythrosine released from the scaffold over time into a range of liquids chosen to approximate physiological conditions, such as saliva or tissue fluid. The fluids were either distilled water-PBS pH 7.4, PBS plus 5% or 10% foetal calf serum (FCS) and a distilled water control. The results for the release of erythrosine from scaffold is shown in FIG. 2. Erythrosine is released at a rate of approximately 7 μg/mg of scaffold over the first 4 days in all three buffered solutions. This is reduced dramatically after the fourth day and between the fourth and eighth day approximately a further 7 μg/mg of scaffold is released. After day eight the scaffold showed visible signs of deterioration with large fractures appearing and sections breaking off. Remaining scaffold had a very lightly pink colour as opposed to a vivid pink colour at the start of the experiment indicating that there was very little erythrosine remaining in the scaffold. In distilled water erythrosine release was initially comparable to that in buffered solution approximately 5 μg/mg of scaffold but following the initial 24 hours dropped significantly to approximately 1 μg/mg of scaffold. When incubated in distilled water for a total of eight days scaffold retained its cohesion as well as much of its colour. Gradual release in all solutions as well as loss of colour being linked to disintegration of the scaffold indicates that erythrosine release is dependant on degradation of the scaffold as a whole. Since erythrosine is readily soluble in water and electrospun scaffolds are very porous any crystallized on the surface of the nano-fibre strands would immediately dissolve indicating that erythrosine has been incorporated into the body of the electrospun fibres with a relatively even distribution.

### EXAMPLE 3

Experiments were conducted to observe and quantify release of erythrosine from scaffold and its diffusion through a gelatinous medium which approximates soft tissue.

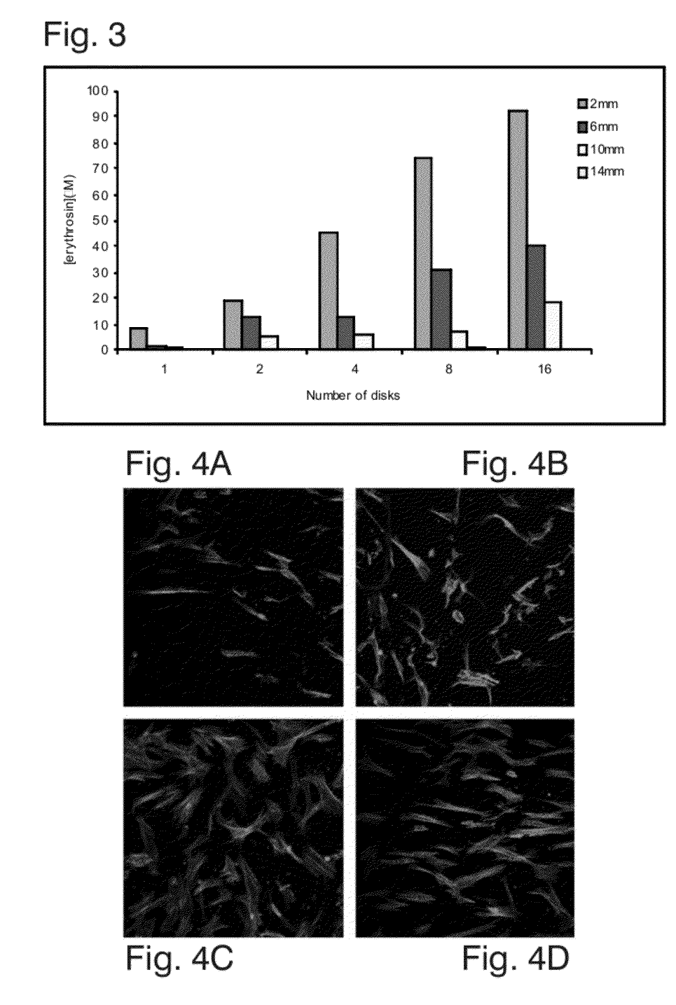

Results showed that erythrosine was released from the scaffold and diffused out into the surrounding agar up to a maximum detected distance of 14 mm (FIG. 3). Although it is postulated that the main site of action is more likely to be very close or in direct contact with the scaffold, it is believed that the ability of erythrosine to diffuse in this way may provide some protection from infection to surrounding healthy tissue.

### EXAMPLE 4

Experiments were conducted to establish the suitability of the scaffold for growth of HDPSCs i.e. to establish that these cells were able to adhere to and proliferate on the scaffold in normal culture conditions. Results shown in FIGS. 4A-D indicate that HDPSCs were able to attach as well as remain viable and proliferate on the scaffold for at least 6 days.

### EXAMPLE 5

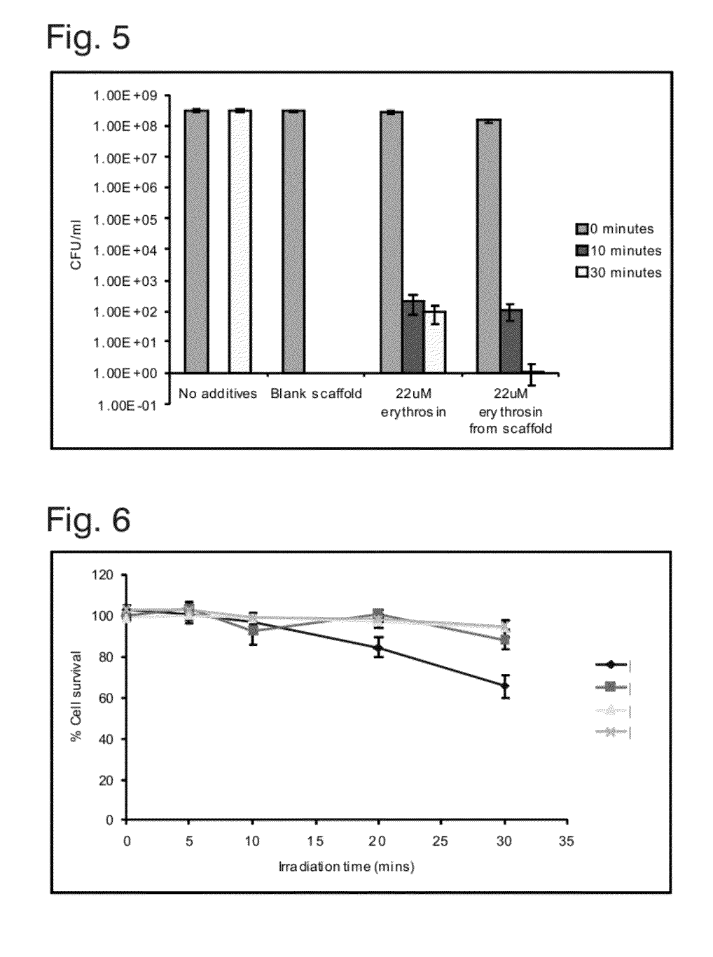

Experiments were conducted to assess the ability of erythrosine contained in the scaffold to act as a photodynamic therapy agent i.e. In the presence of erythrosine from scaffold and on irradiation with visible light bacteria are killed by oxidation of cellular constituents. Irradiation of broth with erythrosine from scaffold induced an 8.1 log₁₀ kill with a 30 minute irradiation and a 6.1 log₁₀ kill with 10 minutes. In comparison erythrosine not taken from scaffold induced a 6.4 log₁₀ and 6.1 log₁₀ kill at 30 and 10 minutes respectively (FIG. 5). Irradiation alone was not sufficient to induce a significant amount of kill. The un-irradiated broth with scaffold-derived erythrosine did have a slightly reduced ability to establish colonies in comparison to un-irradiated broth with non scaffold derived erythrosine, this may be due to enhanced sensitivity of this group to comparatively low intensity light but this has not been confirmed. In summary, scaffold-derived erythrosine retains its ability to act as a PDT agent and shows a 1.5 log₁₀ improvement in comparison to non scaffold derived erythrosine at the 30 minute time point. It is postulated that the improvement in bactericidal effects observed with scaffold-derived erythrosine as compared to non scaffold derived erythrosine is due to a synergistic effect between the scaffold and the erythrosine.

### EXAMPLE 6

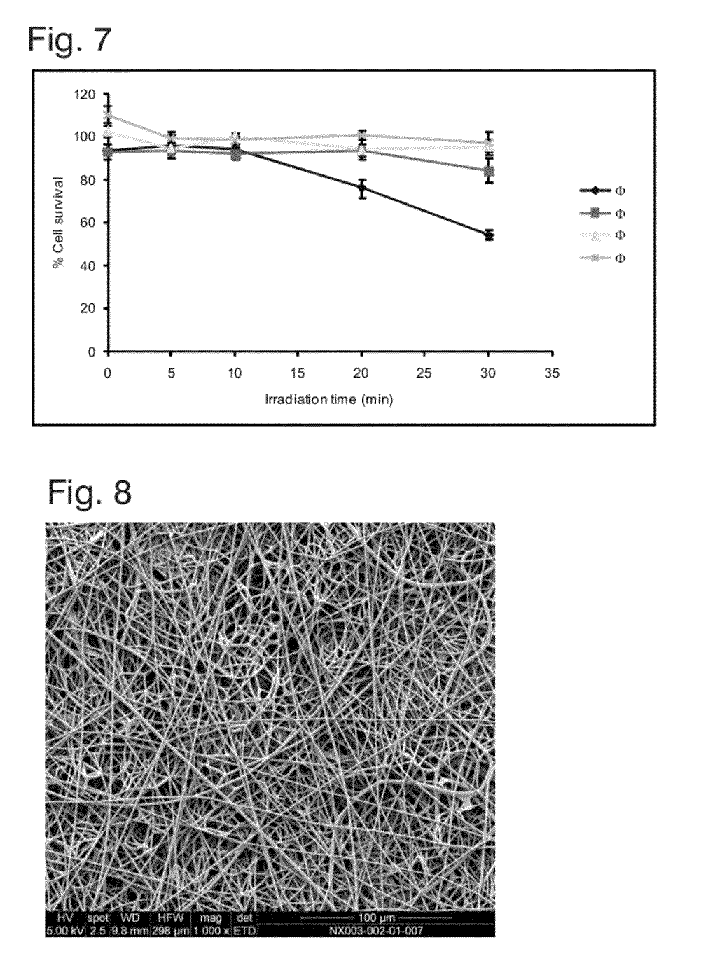

Experiments were conducted to determine the extent to which PDT using erythrosine derived from scaffold kills mammalian cells at concentrations and times sufficient to kill bacteria. Results showed that up to concentrations of 22 μM erythrosine PDT has no significant effect on cell survival irrespective of the source of erythrosine or the irradiation time up to 30 minutes. At a concentration of 44 μM erythrosine PDT does have an effect on cell survival at irradiation times of 20 and 30 minutes. This effect is seen in both groups and to a slightly higher degree from non scaffold-derived erythrosine although this difference is not significant (FIGS. 6 and 7). HDPSCs are not affected significantly at concentrations of erythrosine and irradiation times sufficient to kill bacteria. Moreover, since higher concentrations of erythrosine do not begin to become detrimental until after 10 minutes it may be possible in a clinical setting to use a higher dose of erythrosine for a short period to achieve the desired effect.

### EXAMPLE 7

Experiments were conducted using scaffolds prepared with the alternative bioresorbable polyesters poly(L-lactic acid) (PLLA), polycaprolactone and a copolymer of L-lactic acid and glycolic acid (PLGA 10:90). Experiments were also conducted using scaffolds prepared with the alternative photoactive agents methylene blue, polychrome methylene blue, toluidine blue O, haematoporphyrin IX and chlorin e₆:

The same general method as described previously was used to prepare a 12 w/w % solution of PGA in HFIP containing 5.0 w/w % methylene blue relative to the dry weight of PGA. The same general electrospinning method as described previously was then used to prepare non-woven fibrous scaffolds of PGA containing 5% methylene blue. In this case the needle tip to mandrel distance was set to 90 mm and the voltage was set to 16.0 kV. FIG. 8 shows a scanning electron microscope image of the resulting fibrous PGA scaffold containing 5% methylene blue, the scale bar corresponds to a length of 100 μm. The measured mean fibre diameter is 1.31 μm. FIG. 17 shows a photograph of the scaffold (labelled C).

### EXAMPLE 8

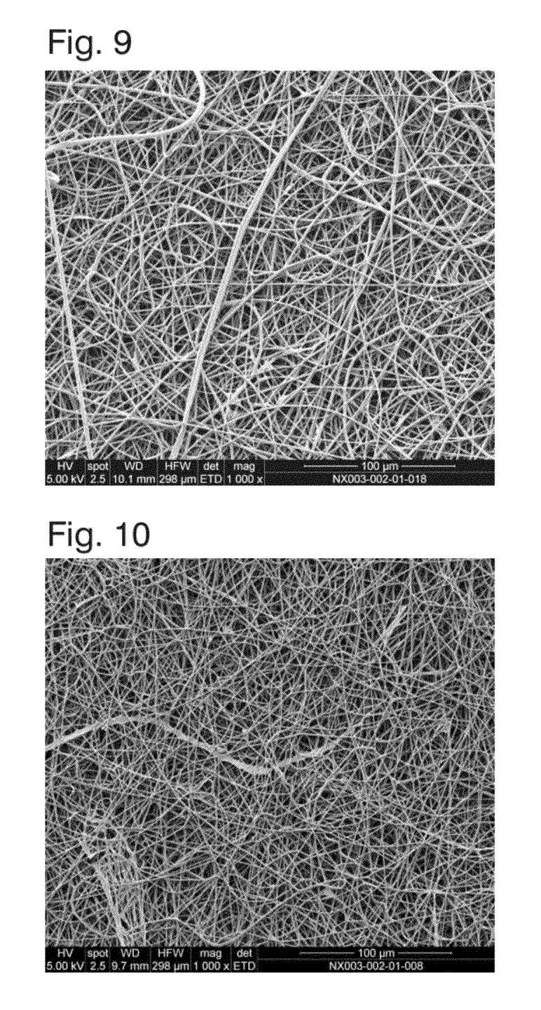

The same general method as described previously was used to prepare a 12 w/w % solution of PGA in HFIP containing 10.0 w/w % methylene blue relative to the dry weight of PGA. The same general electrospinning method as described previously was then used to prepare non-woven fibrous scaffolds of PGA containing 10% methylene blue. In this case the needle tip to mandrel distance was set to 120 mm, the voltage was set to 16.0 kV and the syringe pump rate was 0.04 mLmin⁻¹ per needle. FIG. 9 shows a scanning electron microscope image of the resulting fibrous PGA scaffold containing 10% methylene blue, the scale bar corresponds to a length of 100 μm. The measured mean fibre diameter is 1.23 μm. FIG. 17 shows a photograph of the scaffold (labelled I).

### EXAMPLE 9

The same general method as described previously was used to prepare a 10 w/w % solution of PGA in HFIP containing 5.0 w/w % polychrome methylene blue relative to the dry weight of PGA. The same general electrospinning method as described previously was then used to prepare non-woven fibrous scaffolds of PGA containing polychrome methylene blue. In this case the needle tip to mandrel distance was set to 60 mm, the voltage was set to 16.0 kV and the syringe pump rate was 0.04 mLmin⁻¹ per needle. FIG. 10 shows a scanning electron microscope image of the resulting fibrous PGA scaffold containing polychrome methylene blue, the scale bar corresponds to a length of 100 μm. The measured mean fibre diameter is 0.91 μm. FIG. 17 shows a photograph of the scaffold (labelled D).

### EXAMPLE 10

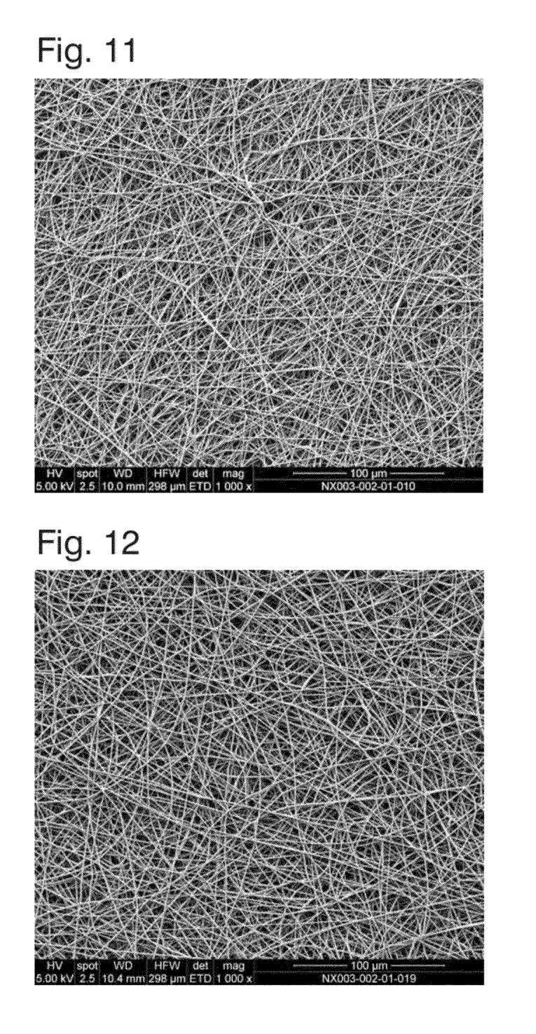

The same general method as described previously was used to prepare a 10 w/w % solution of PGA in HFIP containing 5.0 w/w % toluidine blue O relative to the dry weight of PGA. The same general electrospinning method as described previously was then used to prepare non-woven fibrous scaffolds of PGA containing toluidine blue O. In this case the needle tip to mandrel distance was set to 120 mm, the voltage was set to 15.0 kV and the syringe pump rate was 0.04 mLmin⁻¹ per needle. FIG. 11 shows a scanning electron microscope image of the resulting fibrous PGA scaffold containing toluidine blue O, the scale bar corresponds to a length of 100 μm. The measured mean fibre diameter is 0.74 μm. FIG. 17 shows a photograph of the scaffold (labelled E).

### EXAMPLE 11

The same general method as described previously was used to prepare a 10 w/w % solution of PGA in HFIP containing 5.75 w/w % haematoporphyrin IX relative to the dry weight of PGA. The same general electrospinning method as described previously was then used to prepare non-woven fibrous scaffolds of PGA containing haematoporphyrin IX. In this case the needle tip to mandrel distance was set to 120 mm, the voltage was set to 16.0 kV and the syringe pump rate was 0.04 mLmin⁻¹ per needle. FIG. 12 shows a scanning electron microscope image of the resulting fibrous PGA scaffold containing haematoporphyrin IX, the scale bar corresponds to a length of 100 μm. The measured mean fibre diameter is 0.83 μm. FIG. 17 shows a photograph of the scaffold (labelled J).

### EXAMPLE 12

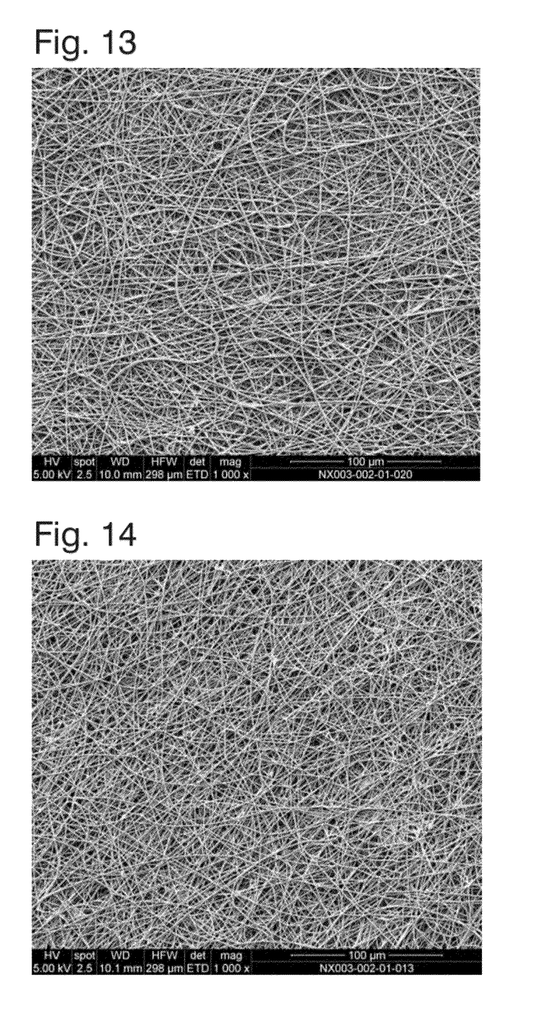

The same general method as described previously was used to prepare a 10 w/w % solution of PGA in HFIP containing 2.15 w/w % chlorin e₆ relative to the dry weight of PGA. The same general electrospinning method as described previously was then used to prepare non-woven fibrous scaffolds of PGA containing chlorin e₆. In this case the needle tip to mandrel distance was set to 120 mm, the voltage was set to 16.0 kV and the syringe pump rate was 0.04 mLmin⁻¹ per needle. FIG. 13 shows a scanning electron microscope image of the resulting fibrous PGA scaffold containing chlorin e₆, the scale bar corresponds to a length of 100 μm. The measured mean fibre diameter is 0.75 μm. FIG. 17 shows a photograph of the scaffold (labelled K).

### EXAMPLE 13

The same general method as described previously was used to prepare a 10 w/w % solution of poly(L-lactic acid-co-glycolic acid) (PLGA 10:90) in HFIP containing 5.0 w/w % methylene blue relative to the dry weight of PLGA. The same general electrospinning method as described previously was then used to prepare non-woven fibrous scaffolds of PLGA containing methylene blue. In this case the needle tip to mandrel distance was set to 120 mm and the voltage was set to 18.0 kV. FIG. 14 shows a scanning electron microscope image of the resulting fibrous PLGA 10:90 scaffold containing methylene blue, the scale bar corresponds to a length of 100 μm. The measured mean fibre diameter is 0.77 μm. FIG. 17 shows a photograph of the scaffold (labelled F).

### EXAMPLE 14

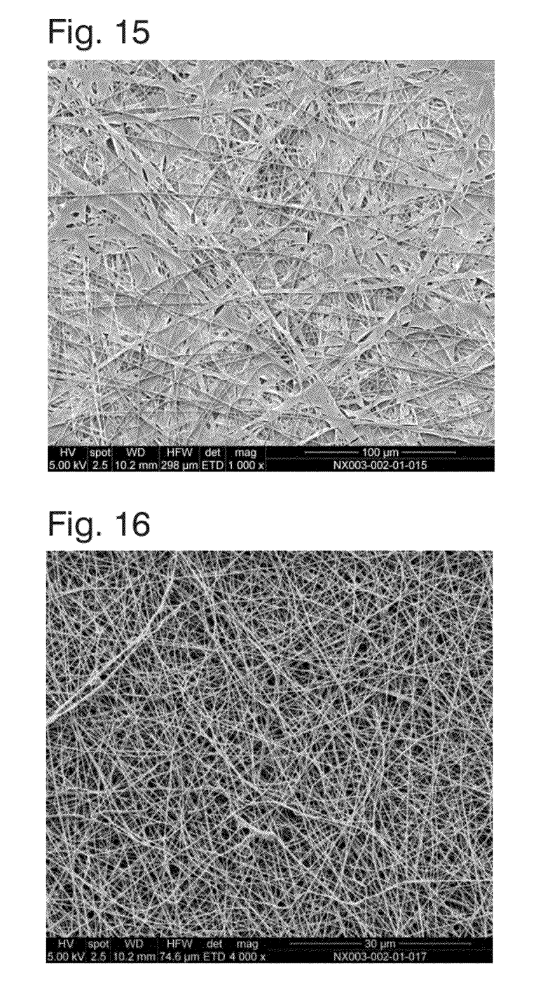

The same general method as described previously was used to prepare an 8 w/w % solution of poly(L-lactic acid) (PLLA) in HFIP containing 5.0 w/w % methylene blue relative to the dry weight of PLLA. The same general electrospinning method as described previously was then used to prepare non-woven fibrous scaffolds of PLLA containing methylene blue. In this case the needle tip to mandrel distance was set to 120 mm, the voltage was set to 16.0 kV and the syringe pump rate was 0.04 mLmin⁻¹ per needle. FIG. 15 shows a scanning electron microscope image of the resulting fibrous PLLA scaffold containing methylene blue, the scale bar corresponds to a length of 100 μm. The measured mean fibre diameter is 0.74 μm. FIG. 17 shows a photograph of the scaffold (labelled G).

### EXAMPLE 15

The same general method as described previously was used to prepare an 8 w/w % solution of polycaprolactone (PCL) in HFIP containing 5.0 w/w % methylene blue relative to the dry weight of PCL. The same general electrospinning method as described previously was then used to prepare non-woven fibrous scaffolds of PCL containing methylene blue. In this case the needle tip to mandrel distance was set to 120 mm, the voltage was set to 25.0 kV and the syringe pump rate was 0.04 mLmin⁻¹ per needle. FIG. 16 shows a scanning electron microscope image of the resulting fibrous PCL scaffold containing methylene blue, the scale bar corresponds to a length of 30 μm. The measured mean fibre diameter is 0.20 μm. FIG. 17 shows a photograph of the scaffold (labelled H).

### EXAMPLE 16

L casei cultures were grown in brain heart infusion media (BHI) containing various additives as shown in Table 1 below. Pieces of blank PGA scaffold and PGA scaffold containing 5 w/w % erythrosine B were incubated overnight in BHI and the scaffold was removed before adding L Casei culture, the erythrosine B concentration was measured and adjusted to 22 μM. For each of the ‘media’ shown in Table 1, two tubes of culture, one tube wrapped in foil, were exposed to light for 30 minutes. After exposure to light, serial dilutions were made of the cultures in BHI which were then grown on Columbian blood agar plates for 48 hours. The number of colonies on the plates was then counted and those having between 30 and 300 colonies were used to calculate the cfu/ml for the cultures. Data show that either light or scaffold alone has little or no effect on bacterial viability. PDT (erythrosine plus light) causes 6 logs of cell kill, whereas in the presence of glycolic acid released from the scaffold (or added to a similar concentration out of a bottle), the amount of cell killing doubles or more. This data indicates there is a synergistic effect between the PDT agent and the alpha hydroxy acid, in this case, glycolic acid.

TABLE 1

| Culture tubes wrapped in | Culture tubes |
|---|---|
| foil and exposed to light (F) | exposed to light (L) |
| BHI | BHI |
| 3.12 × 10⁸ cfu/ml | 3 × 10⁸ cfu/ml |
| BHI + PGA scaffold | BHI + PGA scaffold |
| 2.85 × 10⁸ cfu/ml | 2.85 × 10⁸ cfu/ml |
| BHI + 22 μM Erythrosin B | BHI + 22 μM Erythrosin B |
| 2.75 × 10⁸ cfu/ml | 2.1 × 10² cfu/ml |
| BHI + PGA/Erythrosin B scaffold | BHI + PGA/Erythrosin B scaffold |
| (22 μM erythrosin) | (22 μM erythrosin) |
| 1.4 × 10⁸ cfu/ml | 1.1 × 10² cfu/ml |
| BHI + 22 μM Erythrosin | BHI + 22 μM Erythrosin |
| B + 0.44 mM glycolic acid | B + 0.44 mM glycolic acid |
| 2.4 × 10⁸ cfu/ml | 1.6 × 10¹ cfu/ml |

### EXAMPLE 17

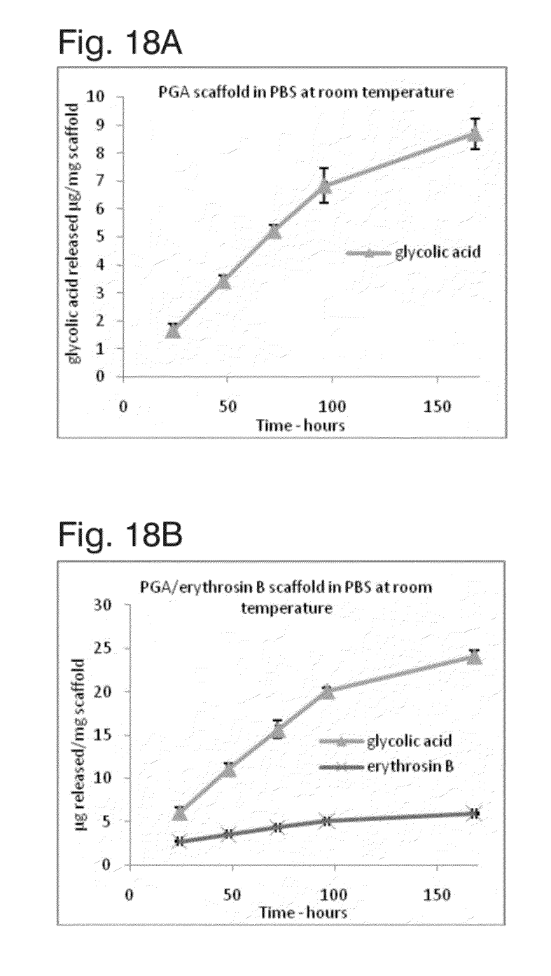

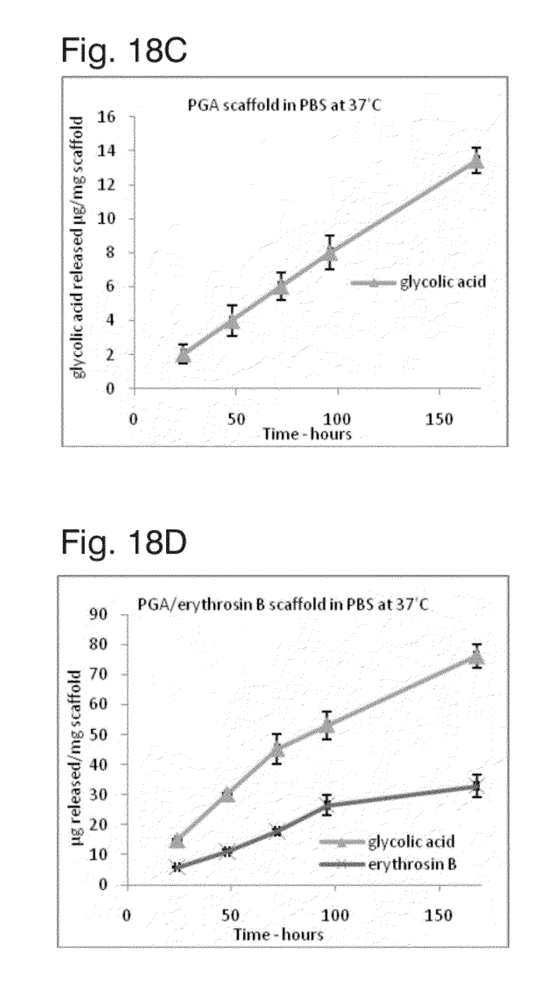

Pieces of blank PGA scaffold and PGA scaffold containing 5 w/w % erythrosine B were incubated in the dark in PBS at room temperature and at 37° C. over a period of 7 days. The concentration of erythrosine B released was measured against a standard at 535 nm after 24, 48, 72, 96 and 168 hours. These solutions were then used to determine the glycolic acid released using a spectrophotometric method taking readings at 480 nm of the colour produced by the reaction of the sample with a beta naphthol reagent. FIG. 18 shows the cumulative release of glycolic acid and of erythrosine B from PGA and PGA/erythrosine B scaffolds. FIG. 18A shows the amount of glycolic acid released and FIG. 18B shows the amount of glycolic acid and erythrosine B released from a PGA scaffold in PBS at room temperature. FIG. 18C shows the amount of glycolic acid released and FIG. 18D shows the amount of glycolic acid and erythrosine B released from a PGA scaffold in PBS at 37° C. Data demonstrates that the release of glycolic acid as the scaffold “dissolves” is mirrored by the curves for the release of erythrosine and suggests that the release of erythrosine is due to dissolution of the scaffold itself, rather than just release of erythrosine that is bound to the surface of the fibres. Data also demonstrates that the release is greater at an elevated temperature (37° C. as compared to room temperature) as dissolution is more rapid. This will have important implications in vivo in calculating and controlling the amount of erythrosine which will be released over a given period of time and the accompanying antibacterial effect.

1. A scaffold comprising fibres that provide a source of at least one alpha hydroxy acid and which encapsulate at least one antimicrobial photoactive agent.

2. A scaffold according to claim 1 wherein the alpha hydroxy acid is selected from the group comprising glycolic acid, lactic acid, citric acid, mandelic acid, tartaric acid, malic acid, and galacturonic acid.

3. A scaffold according to claim 1 wherein the alpha hydroxy acid is glycolic acid.

4. A scaffold according to claim 1 wherein the alpha hydroxy acid is generated by degradation of the fibres or wherein the fibres are coated with or covered in the alpha hydroxy acid.

5. A scaffold according to claim 1 wherein the fibres are composed of a biocompatible polymer or polyester selected from the group comprising poly(glycolic acid) (PGA), poly(L-lactic acid) (PLLA), poly(lactic-co-glycolic acid) (PLGA), polycaprolactone or copolymers, blends and mixtures thereof.

6. A scaffold according to claim 1 comprising a mixture of fibres composed of different polymers or copolymers of different molecular weight.

7. A scaffold according to any preceding claim that has different rates of release of the photoactive agent.

8. A scaffold according to claim 1 further including natural polymer fibres.

9. A scaffold according to claim 1 wherein the at least one photoactive agent incorporated in the scaffold is selected from the group comprising comprising xanthenes, porphyrins, phthalocyanines, chlorins and thiazines or combinations thereof.

10. A scaffold according to claim 9 wherein the photoactive agent is selected from the group comprising erythrosine B, methylene blue, polychrome methylene blue, toluidine blue, haematoporphyrin IX and chlorine e₆ or combinations thereof.

11. A scaffold according to claim 1 wherein the scaffold comprises between 0.1 to 20% w/w of the photoactive agent.

12. A scaffold according to claim 1 wherein the scaffold comprises between 1.0 to 10% w/w of the photoactive agent.

13. A scaffold according to claim 1 wherein the photoactive agent is released from the scaffold in a well-defined manner over a time period which is relevant to infection times in vivo.

14. A scaffold according to claim 1 wherein the fibres comprise PGA fibres and the photoactive agent is selected from the group comprising erythrosine B, methylene blue, polychrome methylene blue, toluidine blue, haematoporphyrin IX and chlorine e₆ or combinations thereof.

15. A scaffold according to claim 1 that is seeded with a population of cells derived from the same or different tissue types.

16. A scaffold according to claim 15 wherein the cells are stem cells or the population contains stem cells.

17. A scaffold according to claim 1 that is a non-woven fabric.

18. A scaffold according to claim 1 wherein the fibres have a mean fibre diameter of between 0.01 to 100.00 microns.

19. A scaffold according to claim 18 wherein the fibres have a mean fibre diameter of between 0.05 to 50.00 microns.

20. (canceled)

21. A scaffold according to claim 1 comprising fibres of the same or different mean average diameter.

22. A scaffold according to claim 1 that is in the form of a sheet, strip, or patch or is deliverable by aerosol or injection so that it is formed in situ at the site of application.

23. A method of manufacturing a scaffold comprising electrospinning a solution comprising a photoactive agent and a biocompatible polymer that hydrolyses to an alpha hydroxy acid, onto a target, wherein the electrospun fibres form a scaffold, optionally the method further comprising the step of seeding the scaffold with a population of cells.

24. (canceled)

25. A method of implanting a scaffold comprising fibres that provide a source of at least one alpha hydroxy acid and which encapsulate at least one antimicrobial photoactive agent, the method including one or more of
(i) a method of delivering a selected population of cells to a tissue comprising implanting the scaffold comprising fibres that provide a source of at least one alpha hydroxy acid and which encapsulate at least one antimicrobial photoactive agent, wherein the scaffold is seeded with the selected population of cells;
(ii) a method of reducing or controlling the risk of a microbial infection following implantation of the scaffold comprising fibres that provide a source of at least one alpha hydroxy acid and which encapsulate at least one antimicrobial photoactive agent, the method further comprising implanting the scaffold at an appropriate site and exposing it to light so as to activate the photoactive agent;
(iii) a method of improving graft or implant survival and/or promoting scaffold integration and/or tissue repair and/or wound healing, the method comprising implanting the scaffold comprising fibres that provide a source of at least one alpha hydroxy acid and which encapsulate at least one antimicrobial agent; and exposing the scaffold to light so as to activate the photoactive agent; and
(iv) a method of controlled release of a photoactive agent at a specified site in or on a human or animal body, the method comprising implanting the scaffold comprising fibres that provide a source of at least one alpha hydroxy acid and which encapsulate at least one antimicrobial photoactive agent, wherein the scaffold is optionally seeded with a selected population of cells.

26. (canceled)

27. A method according to claim 25 that includes the method (ii) and wherein the microbial infection is a bacterial infection.

28. (canceled)

29. A method of restorative dentistry comprising implanting a scaffold at an appropriate site within a buccal cavity, the scaffold comprising fibres that provide a source of at least one alpha hydroxy acid and which encapsulate at least one antimicrobial photoactive agent, the scaffold being seeded with human dental pulp stem cells and exposing said scaffold to light so as to activate the photoactive agent.

30. (canceled)

31. (canceled)

32. A scaffold for delivering a glycolic acid monomer and at least one photoactive agent to a desired site, wherein the glycolic acid monomer is derived as a product of hydrolysis from polymeric fibres within said scaffold or is directly co-administered or is directly derivable from scaffold fibres as the monomer.

33. A scaffold according to claim 11, wherein the scaffold is differentially loaded with photoactive agent in different regions.

34. A scaffold according to claim 16, wherein the cells are human dental pulp stem cells.

35. A method which comprises implanting into a patient a scaffold according to claim 1.
